(19) United States
(12) Patent Application Publication
(10) Pub. No.: US 2023/0355868 A1
(43) Pub. Date: Nov. 9, 2023

(54) INFUSION SYSTEM AND CATHETER FOR SUCH AN INFUSION SYSTEM

(71) Applicant: B. Braun Melsungen AG, Melsungen (DE)

(72) Inventors: Tobias Schroeder, Melsungen (DE); André Weiss, Guxhagen (DE); Jens Wildhagen, Hannover (DE)

(21) Appl. No.: 18/143,577

(22) Filed: May 4, 2023

(30) Foreign Application Priority Data
May 5, 2022 (DE) 10 2022 204 439.8

(51) Int. Cl. A61M 5/142; A61M 5/172; A61M 25/00

(52) U.S. Cl. CPC A61M 5/14228; A61M 5/172; A61M 25/0045; A61M 25/0026

(57) ABSTRACT

An infusion system and method for providing different medical fluids to a patient. The infusion system, which can be used in complex infusion therapies, includes a catheter with a catheter tube and at least one lumen extending longitudinally through the catheter tube between a proximal tube end and a distal tube end. A plurality of pump devices are each connected to the at least one lumen so as to convey fluid. A control device is connected to the pump devices and adapted to control the pump devices so that the different medical fluids can be delivered through the at least one lumen by the pump devices in time division multiplex. The catheter can include the pump devices. Each pump device can be configured as a micropump mounted at the proximal tube end.

### CROSS-REFERENCE TO RELATED APPLICATION(S)

This application claims priority under 35 U.S.C. § 119 to German Application No. 10 2022 204 439.8, filed May 5, 2022, the content of which is incorporated by reference herein in its entirety.

### FIELD

The present disclosure relates to an infusion system for providing different medical fluids to a patient, having a catheter with a catheter tube and at least one lumen, which extends longitudinally through the catheter tube between a proximal tube end and a distal tube end, a plurality of pump devices, which are each connected to the at least one lumen so as to convey fluid, and a control device, which is connected to the plurality of pump devices and is adapted to control the plurality of pump devices in such a way that the different medical fluids can be delivered through the at least one lumen by means of the plurality of pump devices in time division multiplex. The present disclosure furthermore relates to a catheter for such an infusion system.

### BACKGROUND

An infusion system is known, for example, from WO 2019/001879 A1. The known infusion system is adapted to provide a plurality of infusions for a patient, the system comprising a multiplicity of infusion units for administering a multiplicity of medical fluids through an infusion line of an infusion set to the patient, and a control unit for controlling the multiplicity of infusion units. The control unit in this case comprises a multiplex module, which is configured in such a way that it multiplexes (time division multiplex) the multiplicity of medical fluids for multiplexed administration of the medical fluids through the infusion line of the infusion set, the multiplex module comprising a scheduling module which is configured in such a way that it defines at least two packets, each packet comprising at least one medical fluid out of the multiplicity of medical fluids, and in such a way that it arranges the at least two packets in a sequence for the administration of the medical fluids of the at least two packets. The infusion units are connected by means of supply lines to a multiple valve, the multiple valve comprising switchable valves for switching over between the infusions of the various infusion units for supply through a single infusion line connected to the patient. In the known infusion system, unintentional mixing of the medical fluids may take place because of the plurality of supply lines to the multiple valve. Furthermore, because of their size the infusion units are arranged on a frame which is placed next to the patient, in particular next to the patient's bed.

### SUMMARY

It is an object of the present disclosure to provide an infusion system of the type mentioned in the introduction and a catheter for such an infusion system, which offer advantages over the prior art particularly in respect of functionality, structure and/or operating reliability.

In the infusion system according to the present disclosure, the catheter comprises the plurality of pump devices, the pump devices each being configured as a micropump and being mounted at the proximal tube end. The micropumps are preferably mounted at the proximal tube end in such a way that an outlet of each micropump debouches directly into the at least one lumen. In this way, almost no dead volume is created between each micropump and the lumen. The micropumps are preferably integrated into the catheter. In another embodiment, the micropumps are arranged indirectly at but close to the catheter tube. The proximal tube end—in an applied state of the catheter—faces away from the patient, whereas the distal tube end faces toward the patient. The micropumps may in this case correspond to a design suitable therefor, for example the design of a membrane pump, in particular a piezoelectric membrane pump, or a roller pump, also referred to as a peristaltic pump. Depending on the design of the micropump, the plurality of micropumps may be driven and synchronized mechanically and/or electrically in time division multiplex, for example by algorithms for gear control or stepper motors or the like. An individual micropump may have a maximum length of 40 mm, preferably 30 mm, and/or a maximum width of 20 mm, preferably 15 mm, and/or a maximum height of 20 mm, preferably 15 mm. Furthermore, an individual micropump may have a maximum component weight of 30 g, preferably 15 g, particularly preferably 5 g. In addition or as an alternative to the micropumps, in one configuration there are controlled microvalves. During operation of the infusion system, the micropumps are driven in such a way that doses, in particular microdoses, of the various medical fluids are pumped successively into the catheter. Separator fluids may in this case be pumped in between the various medical fluids, for example in order to administer various incompatible infusions successively to the patient. For example, a saline solution may be used as a separator fluid. The control device connected to the plurality of micropumps may furthermore be adapted in such a way that a plurality of micropumps may also be active simultaneously. In this way, it is possible for medical fluids that are mutually compatible and may therefore be mixed with one another to be provided and administered to the patient simultaneously without a separator fluid. The control of the time division multiplex may become as complex as desired. The connection between the control device and the plurality of micropumps may be wireless and/or wired. In one embodiment, the catheter tube has only one lumen, so that the term single-lumen catheter may also be used. In other embodiments, the catheter tube has at least two lumina, so that the term dual-lumen or multi-lumen catheter may also be used. In this way, various incompatible infusions may also be provided simultaneously. If the catheter tube has at least two lumina, there will be a number of catheter supply lines corresponding thereto. The solution according to the present disclosure is suitable in particular for complex infusion therapies. By the use of micropumps which are placed very close to the patient, the overall system is optimized for quasi-continuous infusion of a plurality of infusion solutions, in particular incompatible infusion solutions, with simultaneous prevention of the risks of mixing incompatible infusions. This furthermore allows highly accurate dosing, and undesired bolus or reflux of the medical fluids can be avoided. Said boli or refluxes may, for example, occur as a result of movements of long connecting tubes. Furthermore, highly precise, quasi-continuous medicament provision can be ensured.

In one embodiment of the present disclosure, the plurality of micropumps are each adapted to deliver with a delivery rate of from 0.05 ml/min to 500 ml/min, preferably from 0.1 ml/min to 300 ml/min. For example, there is a delivery rate of up to 300 ml/min in so-called acute dialysis. Catecholamines, for example adrenaline or dopamine, are supplied in diluted form with a flow rate of from 0.1 ml/min. In this embodiment of the present disclosure, the control device is adapted to control the delivery rates. In this way, it is possible to adjust the delivery rate, particularly according to the medicament. By highly accurate microdoses, which are possible in particular only with a very small dead volume between each micropump and the lumen, medicaments may also be supplied quasi-continuously to the patient with a low flow rate. In other embodiments, a plurality of micropumps may be combined with one another. For example, a combination of a micropump having a relatively low delivery rate and a micropump having a relatively high delivery rate makes it possible to obtain the required accuracy for low-dosed medicaments and the required delivery power for high-dosed medicaments. If the micropumps are each configured as a peristaltic pump with a revolving rotor, the respective delivery rate is preferably 2 μl per rotor revolution.

In a further embodiment of the present disclosure, the plurality of micropumps are each adapted for intermittent delivery with a cycle time of between 1 s and 100 s, preferably between 5 s and 20 s. The control device is adapted to control the cycle times. In this way, it is possible to adjust the cycle time, particularly according to the medicament. For critical medicaments, for example adrenaline, the control device may be controlled in such a way that discretely cycled medicament provision acts in a similar way to continuous medicament provision. The micropumps are each activated and deactivated alternately according to the predetermined cycle time in order to deliver a microdose through the catheter. In another configuration of the present disclosure, the cycle time for the intermittent delivery of medicaments, in particular non-critical medicaments, may be more than 100 s.

In a further embodiment of the present disclosure, the control device is adapted to control the delivery rates and/or the cycle times as a function of a predetermined overall delivery rate. The overall delivery rate is made up of the delivery rates existing for the individual micropumps. The control device accordingly controls the delivery rate and/or the cycle time of each individual micropump as a function of the predetermined overall delivery rate. In simplified terms, this embodiment allows a quasi-constant overall delivery rate of the infusion system, that is to say the discrete provision of the individual infusions affects the patient in a similar way to continuous medicament provision because of the very small time interval.

In a further embodiment of the present disclosure, the plurality of micropumps comprise a first micropump for delivering a first medical fluid, a second micropump for delivering a second medical fluid and a third micropump for delivering a third medical fluid. The control device is in this case adapted to control the first, second and third micropumps in such a way that first doses of the first medical fluid and second doses of the second medical fluid can be delivered through the at least one lumen in time division multiplex while being physically separated by means of the third medical fluid. The third medical fluid may preferably be a separator fluid, for example a saline solution. In this way, it is possible to physically separate medical fluids that are mutually incompatible by the separator fluid and nevertheless deliver them in a single lumen. The separator fluid may be delivered while being controlled in a continuously or discretely cycled way. In the case of continuous delivery of the separator fluid, the medical fluids are supplied to the separator fluid and mixed therewith. In this case, the separator fluid temporarily becomes a kind of carrier fluid. The incompatible fluids thereby still remain separated from one another.

In a further embodiment of the present disclosure, at least the first micropump is adapted to reverse a delivery direction. The control device is adapted to control the first micropump in such a way that a first dose of the first medical fluid, provided into the at least one lumen, can be partially aspirated, i.e. sucked, from the at least one lumen by means of a reversal of the delivery direction. By the aspiration of the first medical fluid by means of the first micropump, further provision, in particular unintentional provision, of the first medical fluid into the at least one lumen can be reliably prevented and/or mixing of the first medical fluid with a further medical fluid can be prevented. In this way, it is possible to avoid medical fluids, in particular incompatible medical fluids, already being mixed during the provision into the lumen. During the extraction of the first medical fluid, a separator fluid or the like may additionally be extracted. This additionally counteracts unintentional mixing of incompatible fluids.

In a further embodiment of the present disclosure, the catheter tube has at least one further lumen, the lumen having hydrophobic surface properties and the further lumen having lipophobic surface properties, or vice versa. The surface properties of a lumen are defined by the surface properties of an inner side, delimiting this lumen, of a tube wall of the catheter tube. In the case of a hydrophobic surface consistency, i.e. hydrophobic surface properties, the surface has a low wettability for water-based fluids in the manner of the lotus effect, so that the flow behaviour and/or the delivery rate is promoted for water-based medical fluids because of a minimized drag on the inner side of the catheter tube. In contrast thereto, in the case of a lipophobic surface consistency the surface has a low wettability for fat-based fluids in the manner of the lotus effect, so that the flow behaviour and/or the delivery rate is promoted for fat-based medical fluids because of a minimized drag on the inner side of the catheter tube. In this embodiment of the present disclosure, residue-free delivery of medical fluids is promoted in corresponding use by the surface consistencies of the lumina, so that unintentional mixing of the medical fluids can in turn be prevented.

In a further embodiment of the present disclosure, the catheter has a syringe adapter. For example in emergencies, it is therefore possible to supply a particular medical fluid rapidly and straightforwardly through the syringe adapter to the catheter, and therefore to the patient. The syringe adapter is preferably configured in accordance with a Luer connector, an NRFit connector, a non-Luer connector or the like. The syringe adapter is connected to the at least one lumen so as to convey fluid, and is preferably mounted inseparably on the catheter tube, preferably at its proximal tube end.

In a further embodiment of the present disclosure, the catheter is adapted as a disposable product for single use. In this case, the plurality of micropumps are preferably mounted inseparably at the proximal tube end of the catheter tube. The control device, on the other hand, is adapted for multiple use.

The catheter according to the present disclosure is intended for an infusion system according to the present disclosure and comprises a catheter tube and at least one lumen, which extends longitudinally through the catheter tube between a proximal tube end and a distal tube end. The catheter furthermore comprises a plurality of pump devices, which are each configured as a micropump and are mounted at the proximal tube end. The catheter according to the present disclosure may, for example, be used as a central venous catheter which is introduced into the venous system through a vein of the upper half of the patient's body and the end of which lies in the upper or lower vena cava in front of the right atrium of the heart.

### BRIEF DESCRIPTION OF THE DRAWING FIGURES

Further advantages and features of the present disclosure may be found in the following description of preferred exemplary embodiments that are represented with the aid of the drawings.

FIG. 1 shows a schematically simplified representation of an embodiment of an infusion system according to the present disclosure with an embodiment of a catheter according to the present disclosure and a control device.

FIG. 2 shows a schematically simplified sectional representation of a single-lumen catheter tube of the catheter according to FIG. 1 during delivery of a plurality of medical fluids along a delivery direction.

FIG. 3 shows a schematically simplified sectional representation of a dual-lumen catheter tube as a variant of the catheter tube according to FIG. 2.

FIGS. 4A to 4D each show a schematically simplified sectional representation to illustrate successive steps of the functionality of the infusion system according to FIG. 1.

### DETAILED DESCRIPTION

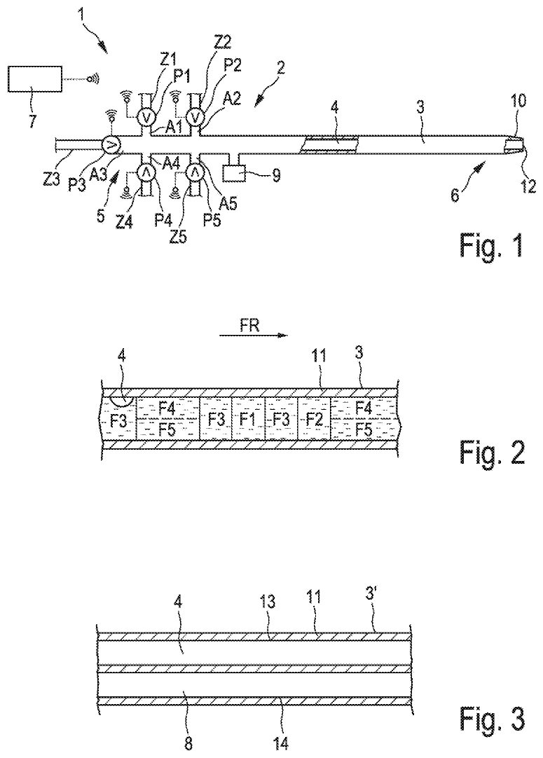

An infusion system 1 according to FIG. 1 is adapted to provide different medical fluids F1 to F5 to a patient. The infusion system 1 is represented in a schematically simplified way and has a catheter 2 and a control device 7. In the embodiment according to FIG. 1, the catheter 2 is configured as a single-lumen catheter and has a catheter tube 3 with a (single) lumen 4. The lumen 4 extends longitudinally through the catheter tube 3 between a proximal tube end 5 and a distal tube end 6.

In alternative embodiments, the catheter is configured as a dual-lumen catheter, as may be seen in FIG. 3. As another alternative, the catheter may have more than two lumina. In the case of multi-lumen catheters, various incompatible infusions may also be provided simultaneously to the patient.

The catheter 2 furthermore has a plurality of pump devices, which are each connected to the lumen 4 so as to convey fluid, as may be seen in FIG. 1. In the exemplary embodiment shown, the catheter 2 has five pump devices.

The specific number of the pump devices is not essential for the underlying present disclosure. In one embodiment which is not represented in the drawings, the catheter 2 has two, three, four or more than five, more than 10 or even more than 20 pump devices.

In the present case, the pump devices are each configured as a micropump P1 to P5 and are mounted at the proximal tube end 5. In the embodiments shown, the micropumps P1 to P5 are each mounted at the proximal tube end 5 in such a way that an outlet A1 to A5 of each micropump P1 to P5 debouches directly into the lumen 4, as may be seen in FIG. 1. In the present case, the micropumps P1, P2, P4 and P5 are arranged in a radial direction with respect to a tube wall 11 of the catheter tube 3, whereas the micropump P3 is arranged in an axial direction with respect to the tube wall 11 of the catheter tube 3 at the proximal tube end 5. In another embodiment, alternatively all the micropumps are arranged in a radial direction with respect to the tube wall of the catheter tube.

In the exemplary embodiment shown according to FIG. 1, the catheter tube 3 has a distal catheter tip 10 at the distal tube end 6. In the present case, the distal catheter tip 10 is an integral part of the catheter tube 3, and to this extent is formed by the distal tube end 6 of the latter. In one embodiment which is not represented in the drawings, the distal catheter tip is configured in the form of a separate component and is assembled firmly with the distal tube end of the catheter tube. The one lumen 4 in the embodiment shown has a distal exit opening 12 in the region of the catheter tip 10, as may be seen in FIG. 1.

The catheter tube 3 is in the present case manufactured in a manner known to a person skilled in the art from a flexible plastic material suitable for medical applications.

The micropumps P1 to P5 are represented in a schematically very simplified manner in the figures. The micropumps P1 to P5 may for example be configured according to the design of a membrane pump or a roller pump, and may be mechanically and/or electrically driven and synchronized according to requirements and the specific application.

The micropumps P1 to P5 have according to FIG. 1 supply lines Z1 to Z5 on the inlet side, which are connected in a manner known per se to infusion bags or the like (not represented in the figures) so as to convey fluid.

The control device 7 is connected to the plurality of micropumps P1 to P5. In the embodiments shown, the control device 7 is connected wirelessly to the plurality of micropumps P1 to P5. In one embodiment which is not represented in the drawings, the control device 7 is connected to the plurality of micropumps P1 to P5 by wires. The control device 7 is adapted to control the plurality of micropumps P1 to P5 in such a way that the different medical fluids F1 to F5 can be delivered by means of the plurality of micropumps P1 to P5 in time division multiplex through the lumen 4 in a delivery direction FR, as may be seen in FIG. 2. In this case, the micropumps P1 to P5 are driven in such a way that predetermined doses, in particular microdoses, of the different medical fluids F1 to F5 are pumped into the catheter 2 at predetermined cycle times. In the embodiments shown, the control device 7 is adapted in such a way that the plurality of micropumps P1 to P5 can also be active simultaneously. In this way, it is possible that a plurality of medical fluids which are mutually compatible, and which may therefore be mixed, can be provided simultaneously even with a single-lumen catheter, as is represented by way of example in FIG. 2 with the medical fluids F4 and F5. In an embodiment with a dual-lumen catheter, different mutually incompatible infusions may thereby also be provided simultaneously to the patient in a respective lumen. In detail, FIG. 2 shows a schematically very simplified sectional representation of the single-lumen catheter tube 3 according to FIG. 1. Individual doses, in particular microdoses, of the plurality of medical fluids F1 to F5 are represented in the catheter tube 3 with a delivery direction FR. In the exemplary embodiment shown, the individual doses of the medical fluids F1 to F3 are delivered successively along the delivery direction FR, whereas the individual doses of the medical fluids F4 and F5 are delivered simultaneously, in particular mixed with one another.

In the embodiments shown, the micropumps P1 to P5 are each adapted for delivery with a delivery rate of from 0.05 ml/min to 500 ml/min, preferably from 0.1 ml/min to 300 ml/min. In the present case, the control device 7 is adapted to control the delivery rates of each individual micropump P1 to P5. With this configuration of the present disclosure, the delivery rate of each micropump P1 to P5 can preferably be set by the control device 7, particularly according to the medicament.

In the embodiments shown, the micropumps P1 to P5 are each adapted for intermittent delivery with a cycle time of between 1 s and 100 s, preferably between 5 s and s. In the present case, the control device 7 is adapted to control the cycle times. With this configuration of the present disclosure, the cycle time of each micropump P1 to P5 can preferably be set by the control device 7, particularly according to the medicament. In other embodiments, the delivery rate of the micropumps P1 to P5 is designed to be controllable, that is to say the delivery quantity of the micropumps P1 to P5 is dependent not only on the cycle length but also on the demand.

In the embodiments shown, the control device 7 is adapted to control the delivery rates and/or the cycle times as a function of a predetermined overall delivery rate. In the present case, the overall delivery rate is made up of the delivery rates of the individual micropumps P1 to P5. The control device 7 preferably controls the delivery rate and/or the cycle time of each individual micropump P1 to P5 as a function of the predetermined overall delivery rate.

In the embodiments shown, the micropumps P1 to P5 comprise a first micropump P1 for delivering a first medical fluid F1, a second micropump P2 for delivering a second medical fluid F2 and a third micropump P3 for delivering a third medical fluid F3. In the present case, the control device 7 is adapted to control the first, second and third micropumps P1, P2, P3 in such a way that first doses of the first medical fluid F1 and second doses of the second medical fluid F2 can successively be delivered through the at least one lumen 4 in time division multiplex while being physically separated by means of the third medical fluid F3, as may be seen particularly in FIG. 2. The third medical fluid F3 is preferably a separator fluid, for example a saline solution. In the exemplary embodiment shown according to FIG. 2, the separator fluid in the form of the third medical fluid F3 is pumped in between the first medical fluid F1 and the second medical fluid F2 and thereby physically separates them from one another. The third medical fluid F3 is in the present case compatible with the first medical fluid F1 and the second medical fluid F2.

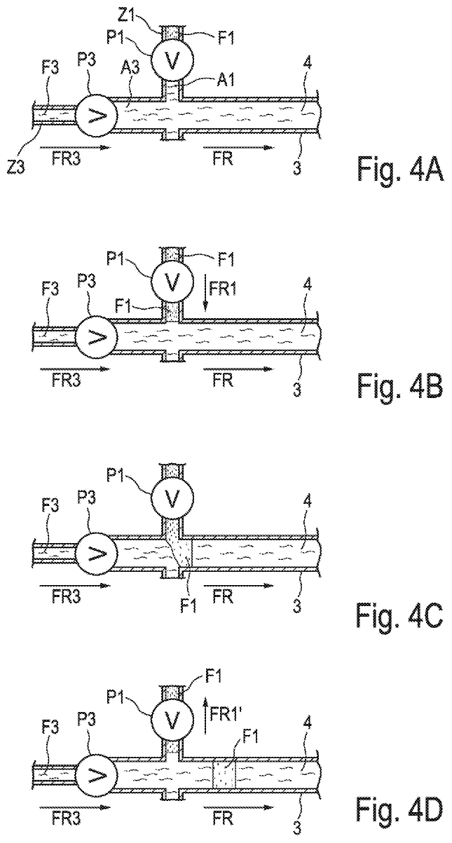

In the embodiments shown, at least the first micropump P1 is adapted to reverse its delivery direction FR1. In the present case, the control device 7 is adapted to control the first micropump P1 in such a way that a first dose of the first medical fluid F1, delivered into the at least one lumen 4, can be partially aspirated, i.e. sucked, from the at least one lumen 4 by means of a reversal of the delivery direction FR1, as is represented in FIG. 4A to 4D.

FIG. 4A to 4D each show a schematically simplified sectional representation to illustrate successive steps of the functionality of the embodiment of the infusion system 1 according to FIG. 1. In the present case, the functionality is described by way of example with the aid of the micropumps P1 and P3. The micropumps P2, P4 and P5 are not represented separately in FIG. 4A to 4D. As already mentioned, the first micropump P1 is adapted to deliver the first medical fluid F1 and the third micropump P3 is adapted to deliver the third medical fluid F3, the third medical fluid F3 being a separator fluid in the present case. In the exemplary embodiment shown, the third micropump P3 is driven in such a way that continuous delivery of the third medical fluid F3 takes place into the lumen 4 in a delivery direction FR3. The first micropump P1 is in the present case time-controlled. In FIG. 4A, the first micropump P1 is deactivated so that it has a delivery rate of 0 ml/min. If the first micropump P1 is briefly activated, it delivers a dose of the first medical fluid F1 along its delivery direction FR1 into the lumen 4, as may be seen in FIG. 4B. In FIG. 4C, the first micropump P1 is again deactivated. The third medical fluid F3 functions as a carrier fluid and entrains at least a part of the delivered dose of the first medical fluid F1 along the delivery direction FR, as may be seen in FIG. 4D. In FIG. 4D, the first micropump P1 is driven in such a way that its delivery direction FR1 is reversed into an opposite delivery direction FR1′. A residual part of the dose of the first medical fluid F1, not yet delivered into the lumen 4, and preferably a small dose of the third medical fluid F3, that is to say in the present case the separator fluid, is thereby aspirated in the opposite delivery direction FR1′, a direction away from the lumen 4. In this way, further entrainment, in particular unintentional entrainment, of the first medical fluid F1 by the third medical fluid F3 or by a further medical fluid, delivered by means of one of the micropumps P2, P4, P5 not separately shown, can be avoided. Mixing of incompatible medical fluids is consequently counteracted particularly effectively.

In the variant according to FIG. 3, the catheter tube 3′ has at least one further lumen 8, the lumen 4 having hydrophobic surface properties and the further lumen 8 having lipophobic surface properties, or vice versa. The surface properties of the lumen 4 are defined by the surface properties of an inner side 13, delimiting this lumen 4, of the tube wall 11 of the catheter tube 3′. Owing to the hydrophobic surface properties of the lumen 4, it has a low wettability for water-based fluids in the manner of the lotus effect, so that the flow behaviour and/or the delivery rate is promoted for water-based medical fluids because of a minimized drag on the inner side 13 of the tube wall 11. The surface properties of the further lumen 8 are defined by the surface properties of an inner side 14, delimiting this further lumen 8, of the tube wall 11 of the catheter tube 3′. Owing to the lipophobic surface properties of the further lumen 8, it has a low wettability for fat-based fluids in the manner of the lotus effect, so that the flow behaviour and/or the delivery rate is promoted for fat-based medical fluids because of a minimized drag on the inner side 14 of the tube wall 11. In the present case, residue-free delivery of medical fluids F1 to F5 is promoted in corresponding use by the different surface properties of the lumina 4, 8, so that unintentional mixing of the medical fluids F1 to F5 can in turn be prevented.

In the embodiments shown, the catheter 2 has a syringe adapter 9, which is represented in a schematically simplified manner in FIG. 1 as a block. The syringe adapter 9 functions as a kind of fluid connector and is connected to the at least one lumen 4 so as to convey fluid. In different embodiments, the syringe adapter 9 may be configured differently, and may for example be a Luer connector, an NRFit connector, a non-Luer connector or the like. In this way, for example in emergencies, it is possible to supply a particular medical fluid rapidly and straightforwardly through the syringe adapter 9 to the catheter 2, and therefore to the patient. In the present case, the syringe adapter 9 is arranged at the proximal tube end 5. In other embodiments, the syringe adapter 9 may be arranged further in the direction of the distal tube end 6.

In the embodiments shown, the catheter 2 is adapted as a disposable product for single use. The control device 7 is preferably adapted for multiple use.

In the present case, the micropumps P1 to P5 are each mounted inseparably at the proximal tube end 5 of the catheter tube 3.

1. An infusion system for providing different medical fluids to a patient, the infusion system comprising:
a catheter comprising a catheter tube and at least one lumen that extends longitudinally through the catheter tube between a proximal tube end and a distal tube end;
a plurality of pump devices, each of the plurality of pump devices being connected to the at least one lumen to convey fluid; and
a control device connected to the plurality of pump devices, the control device being adapted to control the plurality of pump devices so that the different medical fluids are deliverable through the at least one lumen by the plurality of pump devices in time division multiplex,
the catheter comprising the plurality of pump devices, and
the plurality of pump devices comprising a plurality of micropumps, each micropump being mounted at the proximal tube end.

2. The infusion system according to claim 1, wherein the plurality of micropumps are each adapted to deliver with a delivery rate of from 0.05 ml/min to 500 ml/min, and wherein the control device is adapted to control the delivery rate.

3. The infusion system according to claim 2, wherein the control device is adapted to control the delivery rate as a function of a predetermined overall delivery rate

4. The infusion system according to claim 1, wherein the plurality of micropumps are each adapted for intermittent delivery with a cycle time of between 1 s and 100 s, and wherein the control device is adapted to control the cycle time.

5. The infusion system according to claim 4, wherein the control device is adapted to control the cycle time as a function of a predetermined overall delivery rate.

6. The infusion system according to claim 1,
wherein the different medical fluids comprise a first medical fluid, a second medical fluid and a third medical fluid, and
wherein the plurality of micropumps comprise a first micropump for delivering the first medical fluid, a second micropump for delivering the second medical fluid and a third micropump for delivering the third medical fluid,
the control device being adapted to control the first micropump, the second micropump and the third micropump so that first doses of the first medical fluid and second doses of the second medical fluid are deliverable through the at least one lumen in time division multiplex while being physically separated by the third medical fluid.

7. The infusion system according to claim 6, wherein at least the first micropump is adapted to reverse a delivery direction, and the control device is adapted to control the first micropump so that a first dose of the first medical fluid, provided into the at least one lumen, is partially aspirated from the at least one lumen by a reversal of the delivery direction.

8. The infusion system according to claim 1, wherein the at least one lumen comprises a first lumen having hydrophobic surface properties and a second lumen having lipophobic surface properties.

9. The infusion system according to claim 1, wherein the catheter has a syringe adapter.

10. The infusion system according to claim 1, wherein the catheter is adapted as a disposable product for single use, and the control device is adapted for multiple uses.

11. A catheter for an infusion system, the catheter comprising the catheter according to claim 1.
